(19) United States
(12) Patent Application Publication
(10) Pub. No.: US 2026/0241189 A1
(43) Pub. Date: Aug. 20, 2026

(54) Methods and Systems for Identifying and Enhancing Quantum -Like Cognitive Processing in the Human Brain

(71) Applicant: Daniel Glenn Hannay, Ann Arbor, MI (US)

(72) Inventor: Daniel Glenn Hannay, Ann Arbor, MI (US)

(21) Appl. No.: 19/053,459

(22) Filed: Feb. 14, 2025

(51) Int. Cl. A61N 2/00; A61N 7/00

(52) U.S. Cl. CPC A61N 2/002; A61N 2/006; A61N 7/00; A61N 2007/0026

(57) ABSTRACT

A system and method for detecting and enhancing quantum-like cognitive processing in the human brain using neural imaging data analyzed by a machine learning model. The system classifies quantum coherence patterns from EEG, fMRI, or MEG inputs and applies adaptive neurostimulation to enhance cognitive states. Real-time feedback is used to reinforce high-coherence profiles. In certain embodiments, the method supports memory restoration in patients with Alzheimer's or dementia through synchronized stimulation and pharmacological stabilization of microtubule structures.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND

[symbol]

### SUMMARY

[symbol]

### BRIEF DESCRIPTION OF THE DRAWINGS

FIG. 1 illustrates the architecture of a Parallel Universe Quantum Computation (PUQC) system, including mirrored quantum registers, entanglement links, and weak measurement modules.

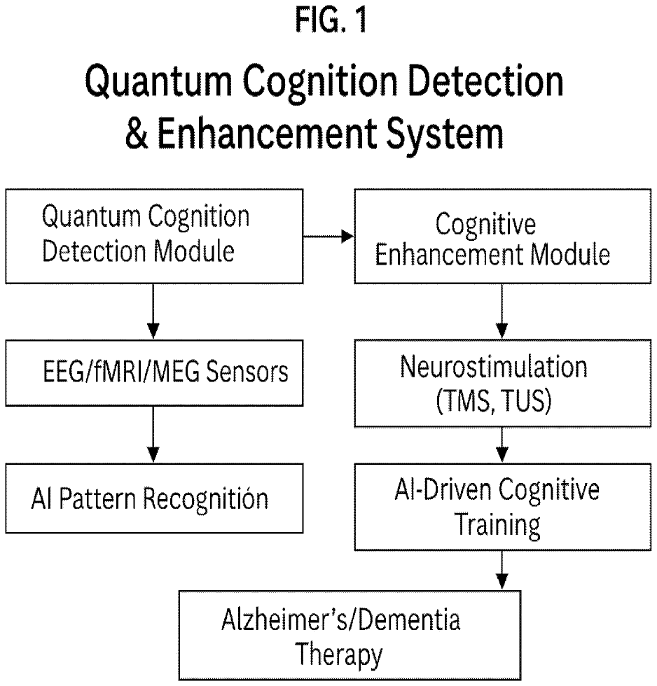

### DETAILED DESCRIPTION

### Continuation of Detailed Description

### Specification

### 1. Background

### 1.1 Limitations of Classical and Conventional Quantum Computing

[symbol] e Classical computing relies on stepwise processing, making complex problem-solving

[symbol] computationally expensive.

[symbol] e Conventional quantum computing (e.g., Grover's Algorithm) reduces complexity but

[symbol] still requires iterative calculations.

### 1.2 The Parallel Universe Computation Hypothesis

[symbol] e lfamirrored universe (U_) exists, it could contain entangled quantum states evolving

[symbol] in reverse time.

[symbol] e A computational process in our universe (U,) could be entangled with its mirrored

[symbol] counterpart in U_, allowing for instantaneous result retrieval.

[symbol] e This mechanism introduces a zero-time computation advantage over conventional

[symbol] quantum systems.

### 2. Summary of the Invention

### 2.1 Overview

This invention proposes a Parallel Universe Quantum Computation System that consists of:
1. Parallel Universe Quantum Entanglement (PUQE): Establishes an entangled link

[symbol] between U, and UL.
2. Mirrored Quantum Registers (MQR): A dual-register quantum system where qubits

[symbol] evolve in opposite time directions.
3. Quantum State Reflection (QSR): An entanglement-preserving transformation that

[symbol] enables information retrieval from UL.
4. Weak Measurement Data Extraction (WMDE): A method for reading precomputed

[symbol] results without decohering entangled states.
5. Superposition-Based Parallel Computation (SBPC): Utilizes the entire parallel

[symbol] universe system to perform multiple computations simultaneously.

### 2.2 Practical Applications

Cryptography: Ultra-fast decryption methods using parallel entangled states.

Al & Machine Learning: Training models at speeds beyond classical quantum methods.

Financial Modeling: Near-instantaneous risk assessment and Monte Carlo simulations.

Physics Simulations: Modeling fundamental particles and high-energy interactions in

[symbol] quantum field theory.

### 3. Detailed Description of the Invention

### 3.1 Quantum Circuit Design for Parallel Universe Computation

A PUQC processor is designed to encode mirrored quantum states such that:

Primary quantum system (U,) evolves forward in time.

Secondary entangled quantum state (U_) evolves in negative time.

Quantum gates in U, mirror inverse-unitary gates in U_, ensuring computational

[symbol] collapse leads to instantaneous solutions.

### 3.2 Operational Mechanism

Step 1: Initialization—A quantum processor entangles a dual-register system linking

[symbol] U, and UL.
Step 2: Processing—Computational operations occur in U, while mirrored

[symbol] transformations occur in U_.
Step 3: Measurement—Collapsing U, retrieves precomputed information from U_,

[symbol] bypassing conventional time constraints.

### 3.3 Hardware Implementation

Superconducting Qubits & Trapped lons: Implementations with scalable quantum

[symbol] processors.

Photonic Quantum Computing: Long-distance entanglement between U, and UL,

[symbol] mitigating decoherence.

1. A system for detecting quantum-like cognition in a human subject, comprising: a neural imaging module configured to collect brain activity data via EEG, fMRI, or MEG; a machine learning model trained on quantum coherence signatures to identify and classify patterns of cognition; and a signal processing module configured to translate imaging data into phase-locked coherence metrics.

2. The system of claim 1, wherein the machine learning model includes reinforcement learning trained on neurotypical and savant-like profiles.

3. The system of claim 1, wherein phase coherence thresholds are used to classify cognitive states in real time.

4. A method of enhancing quantum-like cognition in a human subject, comprising: applying targeted neurostimulation based on detected quantum coherence; and providing neurofeedback in a closed-loop system controlled by an adaptive AI model.

5. The method of claim 4, further comprising: training the AI model using real-time neural coherence data to maximize sustained high-coherence states.

6. A method of improving memory retrieval in patients with Alzheimer's or dementia, comprising: detecting impaired coherence states; applying patterned stimulation to restore synchronization; and administering a pharmacological agent that stabilizes neural microtubule structures.

7. The method of claim 6, wherein the agent comprises a tau-inhibitor or microtubule polymerization stabilizer.
